(19) United States
(12) Patent Application Publication
(10) Pub. No.: US 2007/0225568 A1
(43) Pub. Date: Sep. 27, 2007

(54) SURGICAL RETRACTOR DEVICE AND METHOD OF USE

(76) Inventor: Dennis Colleran, North Attleboro, MA (US)

Correspondence Address: CARR LLP (IST), 670 FOUNDERS SQUARE, 900 JACKSON STREET, DALLAS, TX, 75202, US

(21) Appl. No.: 11/689,881

(22) Filed: Mar. 22, 2007

Related U.S. Application Data
(60) Provisional application No. 60/784,712, filed on Mar. 22, 2006.

(51) Int. Cl. A61B 1/32

(52) U.S. Cl. 600/201

(57) ABSTRACT

Provided is a surgical retractor device and a method for using such a device. In one example, the device includes extension sleeves coupled by wall portions that are at least partially flexible. A void that is open on two ends is defined by the extension sleeves and wall portions. The void may be resized by moving the extensions and/or the wall portions relative to one another.

### CLAIM OF PRIORITY AND CROSS-REFERENCE TO RELATED APPLICATIONS

This application claims priority from U.S. Provisional Patent Application Ser. No. 60/784,712, “SURGICAL RETRACTOR DEVICE AND METHOD OF USE”, filed on Mar. 22, 2006, which is incorporated by reference herein.

This application is related to U.S. patent application Ser. No. 11/362,242, entitled “SURGICAL RETRACTOR APPARATUS AND METHOD OF USE”, filed on Feb. 24, 2006, which claims priority to U.S. Provisional Patent Ser. No. 60/656,125, entitled “SURGICAL RETRACTOR APPARATUS AND METHOD OF USE”, filed on Feb. 24, 2005, both of which are incorporated by reference herein.

### BACKGROUND INFORMATION

The human spine provides a vast array of functions, many of which are mechanical in nature. The spine is constructed to allow nerves from the brain to pass to various portions of the middle and lower body. These nerves, typically called the spinal cord, are located in a region within the spine called the spinal canal. Various nerve bundles emerge from the spine at different locations along the lateral length of the spine. In a healthy spine, these nerves are protected from damage and/or undue pressure thereon by the structure of the spine itself.

The spine has a complex curvature made up of a plurality of individual vertebrae (twenty-four in all) separated by intervertebral discs. These discs hold the vertebrae together in a flexible manner so as to allow relative movement between the vertebrae from front to back and from side to side. This movement allows the body to bend forward and backward, to twist from side to side, and to rotate about a vertical axis. Throughout this movement, when the spine is operating properly, the nerves are maintained clear of the hard structure of the spine.

Over time or because of accidents, the intervertebral discs tend to lose height or become cracked, dehydrated, or herniated. The result is that the height of one or more discs may be reduced, which may lead to compression of the nerve bundles. Such compression may cause pain and, in some cases, damage to the nerves.

Currently, there are many systems and methods at the disposal of a physician for reducing or eliminating the pain by minimizing the stress on the nerve bundles. In some instances, the existing disk is removed and an artificial disk is substituted therefore. In other instances, two or more vertebrae are fused together to prevent relative movement between the fused discs.

Often there is required a system and method for maintaining or recreating proper space for the nerve bundles that emerge from the spine at a certain location. In some cases, a cage or bone graft is placed in the disc space to preserve or restore height and to aid in fusion of the vertebral level. As an aid in stabilizing the vertebrae, one or more rods or braces are placed between the fused vertebrae with the purpose of supporting the vertebrae, usually along the posterior of the spine, while fusion takes place. These rods are often held in place by anchors that are placed into the pedicle of the vertebrae.

Minimally invasive surgical procedures have been developed to fuse the vertebrae. Such procedures can reduce pain, post-operative recovery time, and the destruction of healthy tissue. Generally, a pathological site is accessed through portals rather than through a significant incision, which aids in preserving the integrity of the intervening tissues. Minimally invasive surgical procedures are particularly desirable for spinal and neurosurgical applications because of the need for access to locations deep within the body and the possible range of damage to vital intervening tissues. In such procedures, however, it may be necessary to hold the edges of an incision apart to provide a clear operating field within which the surgeon can operate.

What is needed, therefore, is a tool or retractor adapted to work with minimally invasive procedures that allows the surgeon to have a clear path to the operating field, and a method for using such a tool or retractor.

### SUMMARY

In one embodiment, a retractor comprises first and second wall portions and first and second extension sleeves. The first wall portion has a first proximal end, a first distal end, first and second edges, a first interior surface, and a first exterior surface, wherein at least a portion of the first wall portion is formed of a flexible material. The second wall portion has a second proximal end, a second distal end, third and fourth edges, a second interior surface, and a second exterior surface, wherein at least a portion of the second wall portion is formed of a flexible material, and wherein the second wall portion is positioned so that the second interior surface faces the first interior surface. The first extension sleeve is coupled to the first and second wall portions, wherein a longitudinal axis of the first extension sleeve extends from each of the first and second proximal ends to each of the first and second distal ends, respectively. The second extension sleeve is coupled to the first and second wall portions, wherein a longitudinal axis of the second extension sleeve extends from each of the first and second proximal ends to each of the first and second distal ends, respectively. The first extension sleeve is movable relative to the second extension sleeve from a first position to a second position to resize a void formed by the first and second wall portions and first and second extension sleeves, the void being open at the first and second proximal ends and the first and second distal ends.

In another embodiment, a retractor comprises first and second wall portions formed at least partially from a flexible material, wherein each of the first and second wall portions includes a proximal end and a distal end. The retractor also includes first and second extension sleeves coupled to each of the first and second wall portions, wherein a void defined by the first and second wall portions and the first and second extension sleeves is open at the first and second proximal ends and the first and second distal ends, and wherein the first extension sleeve is movable relative to the second extension sleeve from a first position to a second position to resize the void.

In yet another embodiment, a kit comprises a retractor and a spreader. The retractor has first and second wall portions formed at least partially from a flexible material, wherein each of the first and second wall portions includes a proximal end and a distal end, and first and second extension sleeves coupled to each of the first and second wall portions, wherein a void defined by the first and second wall portions and first and second extension sleeves is open at the first and second proximal ends and the first and second distal ends, and wherein the first extension sleeve is movable relative to the second extension sleeve from a first position to a second position to resize the void. The spreader is sized for insertion into the void, wherein the spreader includes first and second gripping surfaces for engaging the first and second wall portions or the first and second extension sleeves, and first and second handles coupled to the first and second gripping surfaces, respectively, wherein the first handle is movable relative to the second handle to alter a distance between the first and second gripping surfaces.

In still another embodiment, a kit comprises a deformable mesh body having a proximal end and a distal end, wherein a void formed by the mesh body has openings at the proximal and distal ends, and wherein the opening at the distal end is narrower than the opening at the proximal end. In other embodiments the opening at the distal end may be the same size or larger than the opening at the proximal end. The kit also includes a spreader sized for insertion into the mesh body, wherein the spreader includes first and second gripping surfaces for engaging the mesh body and first and second handles coupled to the first and second gripping surfaces, respectively, wherein the first handle is movable relative to the second handle to alter a distance between the first and second gripping surfaces.

In another embodiment, a method comprises inserting a retractor into a surgical opening and separating a first extension sleeve of the retractor from a second extension sleeve of the retractor to enlarge a void defined by the retractor in a first direction, wherein the separating is achieved by applying a force necessary to stretch first and second walls formed at least partially of a flexible material, wherein the first and second walls join the first and second extension sleeves.

### BRIEF DESCRIPTION OF THE DRAWINGS

FIG. 1 is a perspective view illustrating one embodiment of a retracting device.

FIG. 2 is a side view of the retracting device of FIG. 1 in a first or collapsed position.

FIG. 3 is a top view of the retracting device of FIG. 1 in a first or collapsed position.

FIG. 4 is a side view of the retracting device of FIG. 1 in a second or expanded position.

FIG. 5 is a top view of the retracting device of FIG. 1 in a second or expanded position.

FIG. 6 is a perspective view illustrating another embodiment of a retracting device.

FIG. 7 is a perspective view illustrating yet another embodiment of a retracting device.

FIG. 8a is a perspective view illustrating still another embodiment of a retracting device.

FIG. 8b is a perspective view of the retracting device of FIG. 8a with one wall in an expanded position.

FIG. 9a is a perspective view illustrating still another embodiment of a retracting device.

FIG. 9b is a perspective view of the retracting device of FIG. 9a in an expanded position.

FIG. 10 is a perspective view of the retracting device of FIG. 9a in an expanded position.

FIG. 11 is a perspective view illustrating still another embodiment of a retracting device.

FIGS. 12a-12d illustrate various embodiments of a holder that may be used with the retracting device of FIG. 11.

FIGS. 13a-13c illustrate various embodiments of spreaders that may be used with a retracting device.

FIG. 13d illustrates one embodiment of using a retractor.

FIGS. 14a-14f illustrate various mesh patterns that may be used in a retracting device.

FIG. 15 illustrates one embodiment of an encased mesh that may be used in a retracting device.

FIG. 16 illustrates another embodiment of an encased mesh that may be used in a retracting device.

### DETAILED DESCRIPTION

The present disclosure is directed to systems and methods for retracting tissues in surgical procedures. It is understood that the following disclosure provides many different embodiments or examples. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

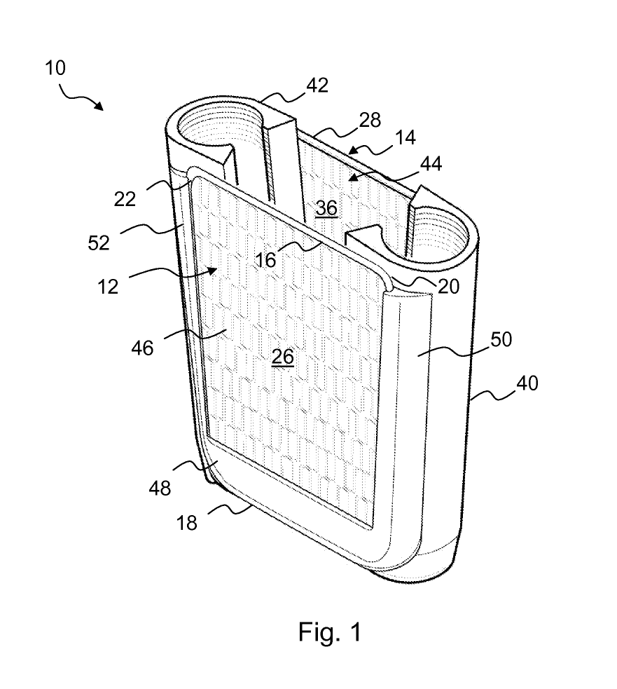

Referring to FIG. 1, a perspective view of one embodiment of a retractor 10 is provided. As illustrated, the retractor 10 may include a wall 12 and a wall 14. The wall 14 may be positioned at a predetermined distance from the wall 12 in a generally parallel manner. The wall 12 may include a proximal end 16, distal end 18, two sides 20 and 22, an interior surface 24 (not shown), and an exterior surface 26. The wall 14 may include a proximal end 28, distal end 30 (not shown), two sides 32 and 34 (not shown), an interior surface 36, and an exterior surface 38 (not shown).

In the present example, the predetermined distance separating the walls 12 and 14 may be defined by extension sleeves 40 and 42, which space the walls apart to create a void 44. The void 44 may be placed over a surgical area to allow access to the surgical site. Each of the extension sleeves 40 and 42 may be coupled to one or both of the walls 12 and 14 using a variety of different couplings, such as the grooved couplers illustrated in FIG. 1 and described in greater detail below. In some embodiments, the extension sleeves 40 and 42 may be tapered at the distal end of the retractor 10.

In some embodiments, at least a portion of one or both of the walls 12 and 14 may be formed of a flexible material, such as mesh, wire, or polymer material. For purposes of convenience, the term “mesh” is used throughout the disclosure to refer to any such flexible material. The mesh may be selected to provide a desired level of rigidity while still being deformable within certain parameters. For example, the mesh may have a level of rigidity that requires application of a certain amount of force in order for the mesh to be expanded or collapsed. Accordingly, when expanded, the mesh may be rigid enough to resist collapsing under the pressure exerted by the surrounding tissue, while still maintaining a level of malleability that allows the mesh to be expanded/collapsed when acted upon by external forces different from and/or larger than the pressure exerted by the surrounding tissue. In some embodiments, the mesh may be made from a shape memory alloy that reacts to the application of heat, electricity, or other means to enable the retractor to take on its original shape to ease removal from the body.

The mesh may allow for a better distribution of forces on the tissue than a similarly shaped rigid wall, and may thereby decrease stress and potential damage to the tissue during retraction. As will be described in greater detail below, the mesh portion of the wall may be formed using various mesh patterns and thicknesses and may be formed to have a desired level of malleability. The mesh may enable the retractor to be inserted with a small form factor and then expanded to an infinite number of larger free formed shapes. In some embodiments, the retractor may have one or more rigid panels/splines/channels. Such structural elements may aid in holding the retractor in place, may allow the holding of other devices by the retractor, and/or may provide more stiff regions to the retracted area.

In the present embodiment, the wall 12 may include a mesh portion 46 that is encased in a polymer or elastomer (e.g., the mesh portion may be placed between two sheets of polymer or elastomer or the mesh portion may be molded within) to protect the tissue during use. It is understood that other embodiments may exist where each individual wire is encased in polymer or elastomer. The wall 12 may also include a protective leading edge 48 made, for example, of polymer/elastomer having an increased thickness. The wall 14 may be similarly or differently configured.

With continued reference to FIG. 1, the extensions 40 and 42 includes grooved couplers 50 and 52, respectively, that may run generally parallel to the extensions 40 and 42. The couplers 50 and 52 enable the extensions 40 and 42 to fasten to the wall 12. In the present example, the coupler 50 includes a groove for receiving the side 20 of the wall 14, and the coupler 52 includes a groove for receiving the side 22. It is understood that the couplers 50 and 52 and their grooves are for purposes of illustration and that many other structures may be used to fasten the walls 12 and 14 to the extensions 40 and 42. For example, the walls 12 and 14 and/or the couplers 50 and 52 may be slidingly coupled to the extensions 40 and 42 using fasteners as disclosed in previously incorporated U.S. patent application Ser. No. 11/362,242, entitled “SURGICAL RETRACTOR APPARATUS AND METHOD OF USE”, filed on Feb. 24, 2006.

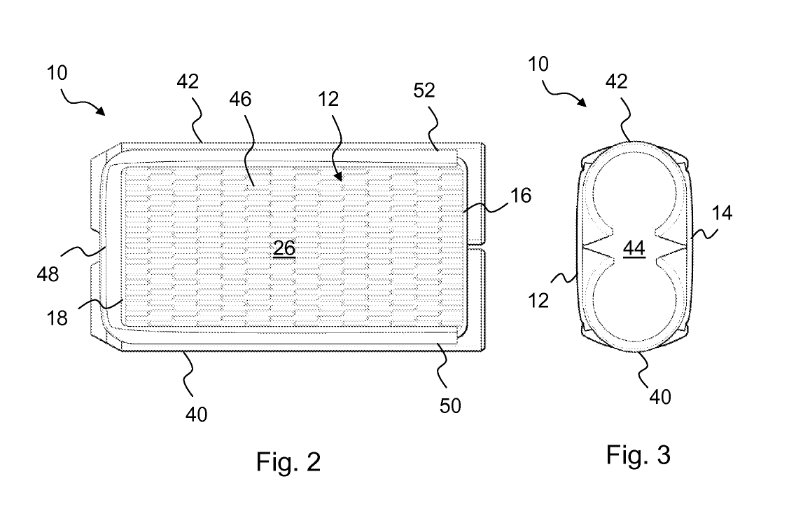

Referring to FIG. 2, a side view of the retractor 10 of FIG. 1 is illustrated in a first or collapsed position. In this embodiment, the wall 12 may be generally rectangular in shape, although it is understood that the wall may have other thicknesses and/or shapes, such as trapezoidal or triangular. In the illustration provided by FIG. 2, portions of the extension sleeves 40 and 42 may extend from the wall 12.

Referring to FIG. 3, a top view of the retractor 10 of FIG. 1 is illustrated in the first or collapsed position.

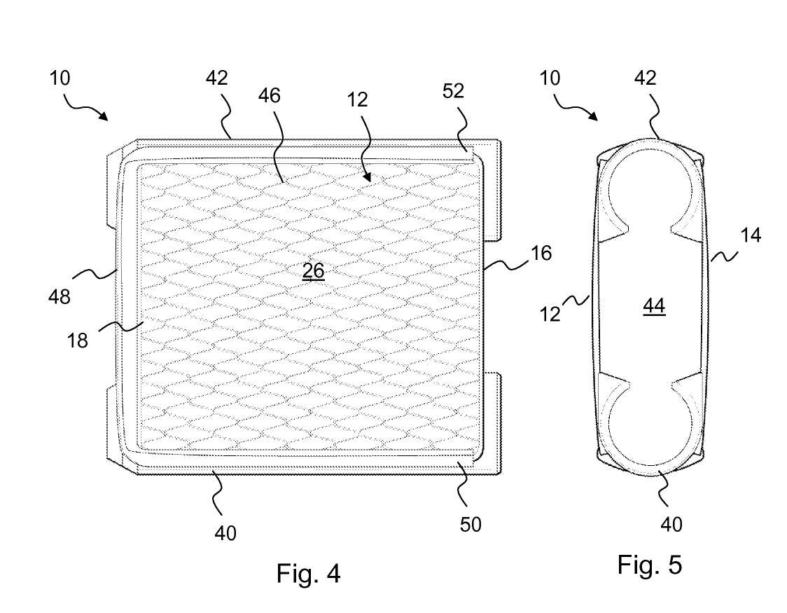

Referring to FIG. 4, a side view of the retractor 10 of FIG. 1 is illustrated in a second or expanded position. In the expanded position, the mesh portion 46 of the wall 12 may be partially or completely extended to enlarge the void 44 (FIG. 1). Due to the properties of the mesh portion 46, the retractor 10 may remain in the expanded position until the mesh portion is collapsed by an outside force (e.g., a user).

Referring to FIG. 5, a top view of the retractor 10 of FIG. 1 is illustrated in the second or expanded position.

Referring to FIG. 6, in another embodiment, the retractor 10 is illustrated with the wall 12 in an expanded position (e.g., moved away from the wall 14). As illustrated, the exterior surface 26 of the wall 12 may be forced outward independently of the wall 14 to enlarge the void 44. Although not shown in an expanded state, the wall 14 may be similarly expandable.

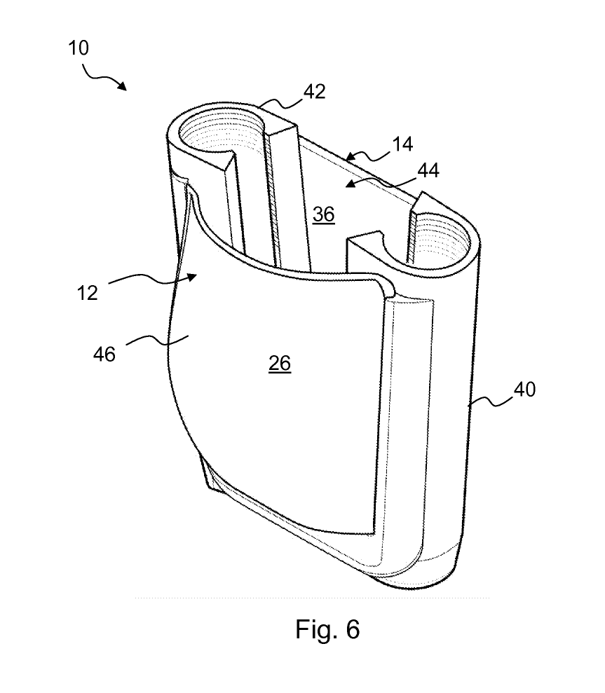

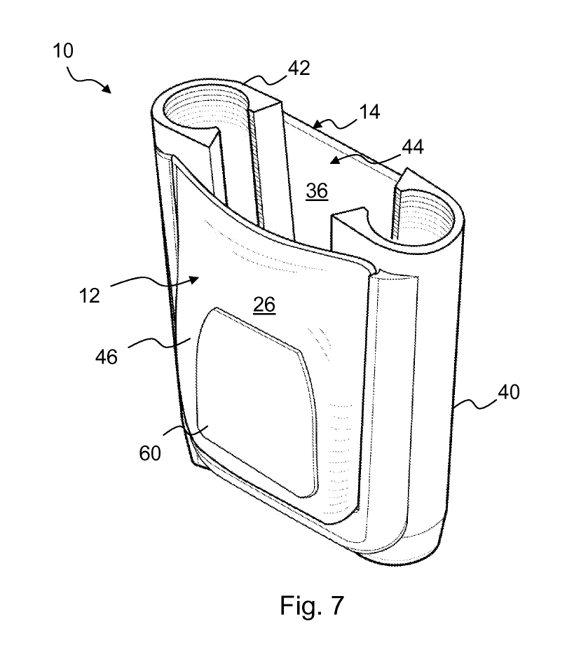

Referring to FIG. 7, in yet another embodiment, the retractor 10 is illustrated with a rigid or semi-rigid plate 60 affixed to the exterior surface 26 of the wall 12, which shown in an expanded position (e.g., moved away from the wall 14). The plate 60 may aid in holding the retractor 10 in an expanded position and/or may allow the coupling of other devices (e.g., a light source) to the retractor. As illustrated, the exterior surface 26 of the wall 12 may be forced outward independently of the wall 14 to enlarge the void 44. The plate 60, which may be substantially rigid in the present embodiment, may force the exterior surface 26 to conform to a desired shape. In some embodiments, the plate 60 may establish a minimum collapsed size by ensuring that there is some separation (e.g., the width of the plate 60) between the extensions 40 and 42. It is understood that the plate 60 may be located in various positions, including on the exterior surface 26, interior surface 24, or integrated within the wall 12. Although not shown, a similar panel may be coupled to the wall 14.

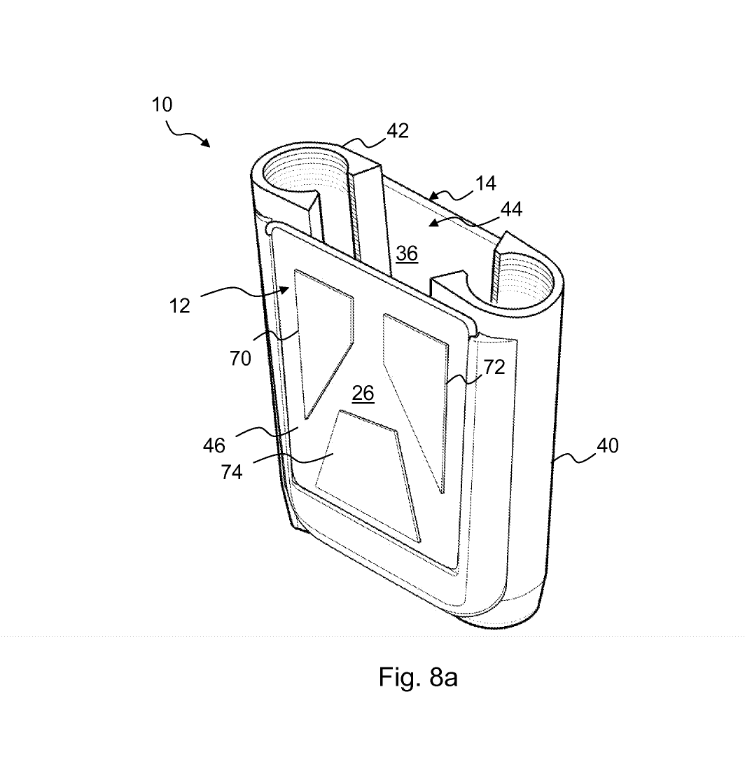

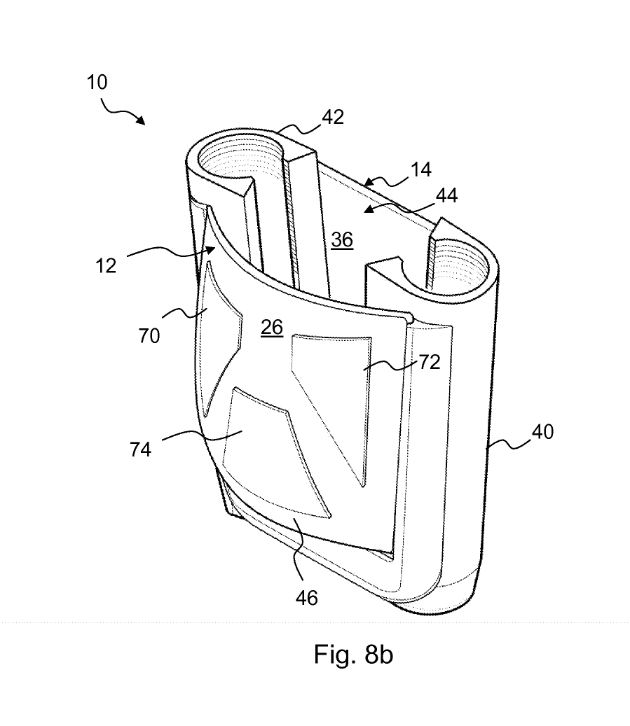

Referring to FIGS. 8a and 8b, in still another embodiment, the retractor 10 is illustrated with multiple rigid or semi-rigid plates 70, 72, and 74 affixed to the exterior surface 26 of the wall 12. As illustrated in FIG. 8a, the plates 70, 72, and 74 are substantially flat and may establish a minimum collapsed size by ensuring that there is some separation between the extensions 40 and 42. The plates 70, 72, and 74 may aid in holding the retractor 10 in an expanded position and/or may allow the coupling of other devices (e.g., a light source) to the retractor. As illustrated in FIG. 8b, the exterior surface 26 of the wall 12 may be forced outward independently of the wall 14 to enlarge the void 44. The plates 70, 72, and 74, which may be semi-rigid in the present embodiment, may provide additional rigidity to the mesh 46 while conforming to the expanded shape of the mesh. Alternatively, the plates 70, 72, and 74 may be rigid. It is understood that the plates 70, 72, and 74 may be located in various positions, including on the exterior surface 26, interior surface 24, or integrated within the wall 12. Although not shown, one or more similar panels may be coupled to the wall 14.

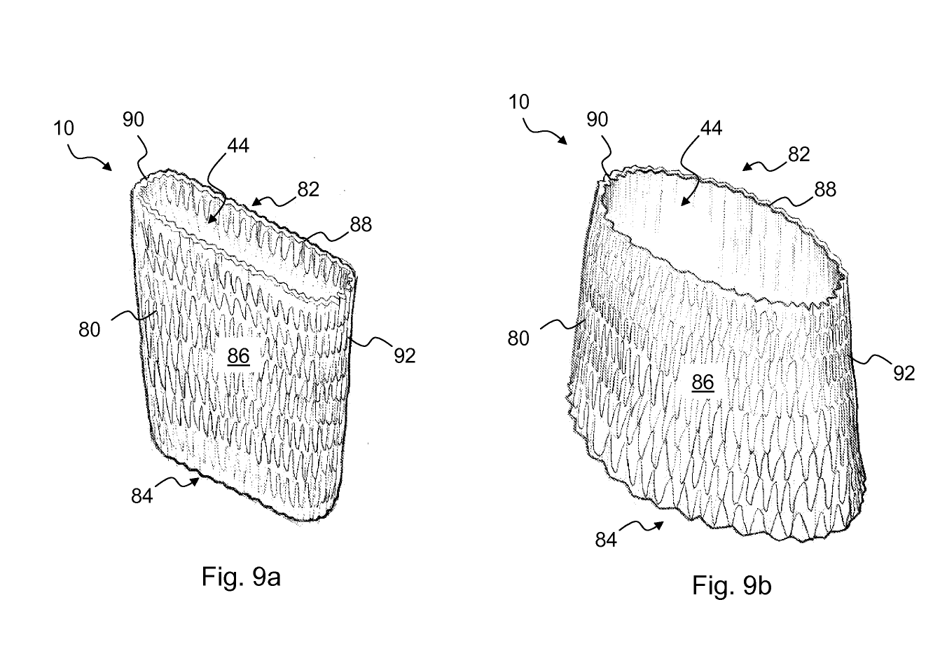

Referring to FIGS. 9a and 9b, in another embodiment, the retractor 10 is illustrated as being formed from a mesh material 80. As illustrated in FIG. 9a, the mesh may be formed as having a generally trapezoidal shape when viewed from the side. A proximal end 82 of the mesh may be generally parallel with a distal end 84. Other embodiments are also contemplated wherein the mesh when viewed on end may have a cross-section with a substantially race track shape, a generally trapezoidal shape, a substantially elliptical shape, or a substantially cylindrical shape. Two walls 86 and 88 may be coupled by sides 90 and 92 to form the void 44. The retractor 80 may be a single piece of material or may comprise multiple mesh pieces coupled together. In the present example, the two sides 90 and 92 may slope from the proximal end 82 towards the distal end 84, providing a shape that is narrower at the distal end than at the proximal end when the retractor is an a collapsed position. As illustrated in FIG. 9b, the mesh 80 may be deformed as desired (within the constraints imposed by the mesh material) to form an expanded shape. Accordingly, the shape of the retractor 10 may be any shape that may be formed by the mesh 80.

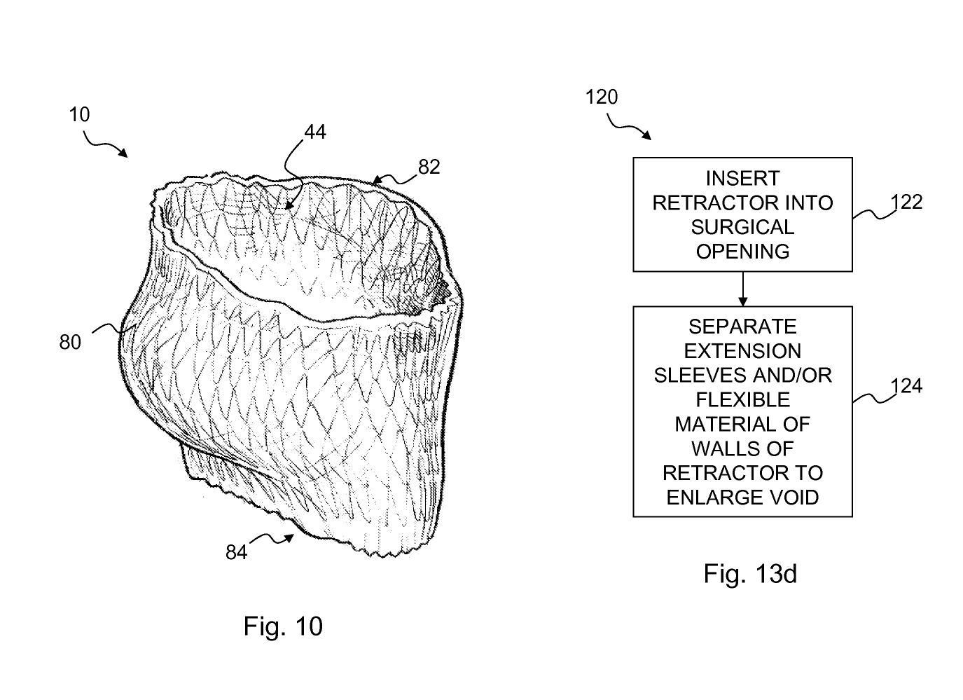

FIG. 10 illustrates another embodiment of the expanded retractor 10 of FIG. 9a. In the present example, a bulge has been formed near to the proximal end 82, while the opening at the distal end 84 is smaller than the bulging area and the opening at the proximal end.

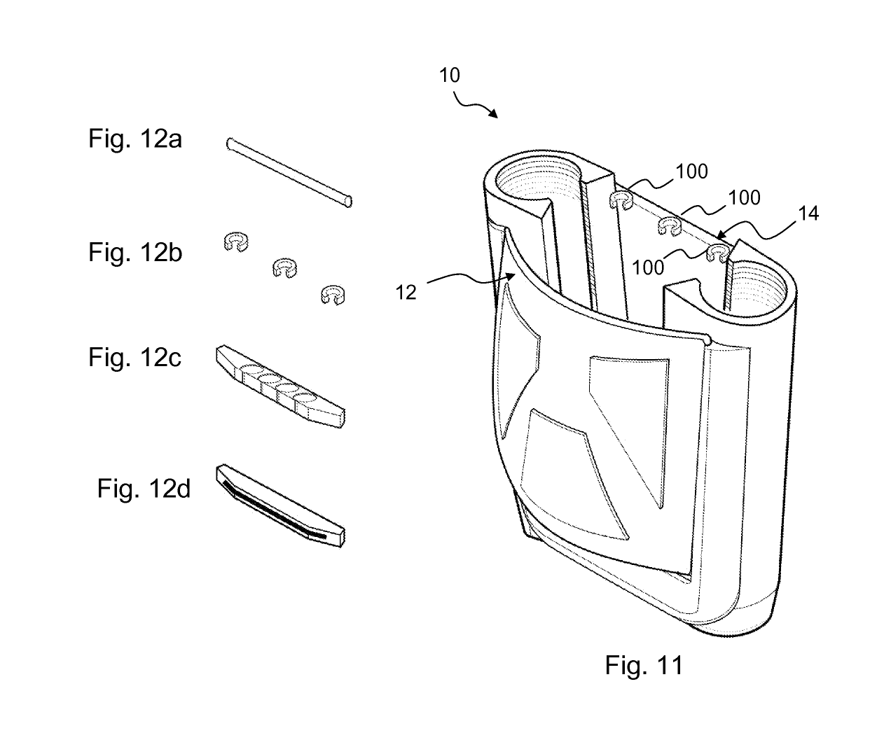

Referring to FIG. 11 and with additional reference to FIGS. 12a-12d, in still another embodiment, the retractor 10 may include one or more holders 100. As illustrated in FIG. 11, the holders 100 may be attached to one of the walls 12 and/or 14. The particular shape and/or location of a holder 100 may depend on its intended use. For example, one holder 100 may be used to couple a light (e.g., an optic fiber) to the retractor 10, while another holder 100 may be used to couple a surgical instrument to the retractor. Furthermore, in addition to the exemplary holders illustrated in FIGS. 12a-12d, a holder may include a slot, hook, window, rail, or any other protrusion and/or recess.

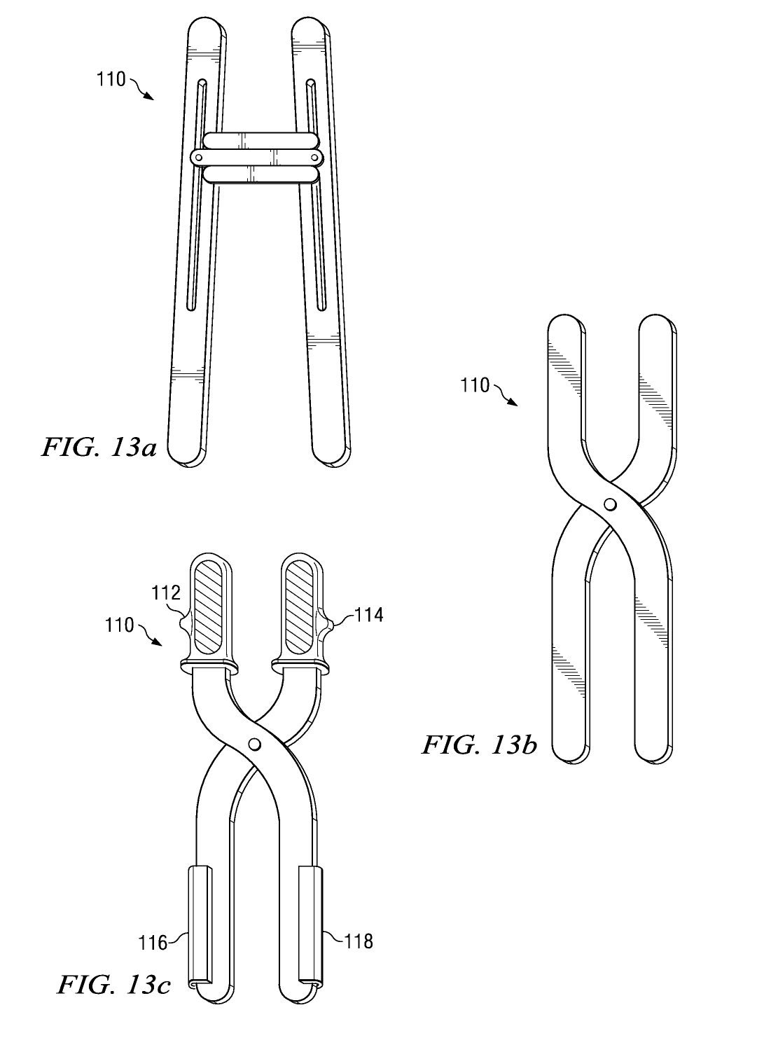

Referring to FIGS. 13a-13c, various embodiments of a spreader 110 are illustrated. The spreader 110 may be used with one or more of the previously discussed embodiments of the retractor 10 or with another retractor to expand the retractor from a collapsed position to an expanded position. For example, the spreader 110 may be inserted into the retractor 10 and actuated to apply force to the interior of the retractor. In some embodiments, such as that illustrated in FIG. 13c, the spreader 110 may have a relatively small form factor on entry into the retractor 10, and then may expand when two handles 112 and 114 are squeezed together. Gripping surfaces 116 and 118 (FIG. 13c) may also be used to prevent the spreader 110 from slipping on the interior surfaces of the retractor 10. In some embodiments, the spreader 110 may couple to the interior of the retractor 10 or may be attached to the exterior of the retractor in order to apply pressure to collapse the retractor.

Referring to FIG. 13d, one embodiment of a method 120 illustrates a process by which a retractor, such as the retractor 10 of FIG. 1, may be expanded using a spreader, such as one of the spreaders 100 of FIGS. 13a-13c. In step 122, the retractor 10 is inserted into a surgical opening. In step 124, a first extension sleeve of the retractor 10 is separated from a second extension sleeve of the retractor to enlarge a void defined by the retractor in a first direction. The separating may be achieved by applying a force necessary to stretch first and second walls formed at least partially of a flexible material, wherein the first and second walls join the first and second extension sleeves. In other embodiments, the spreader may be used to separate the walls in a second direction.

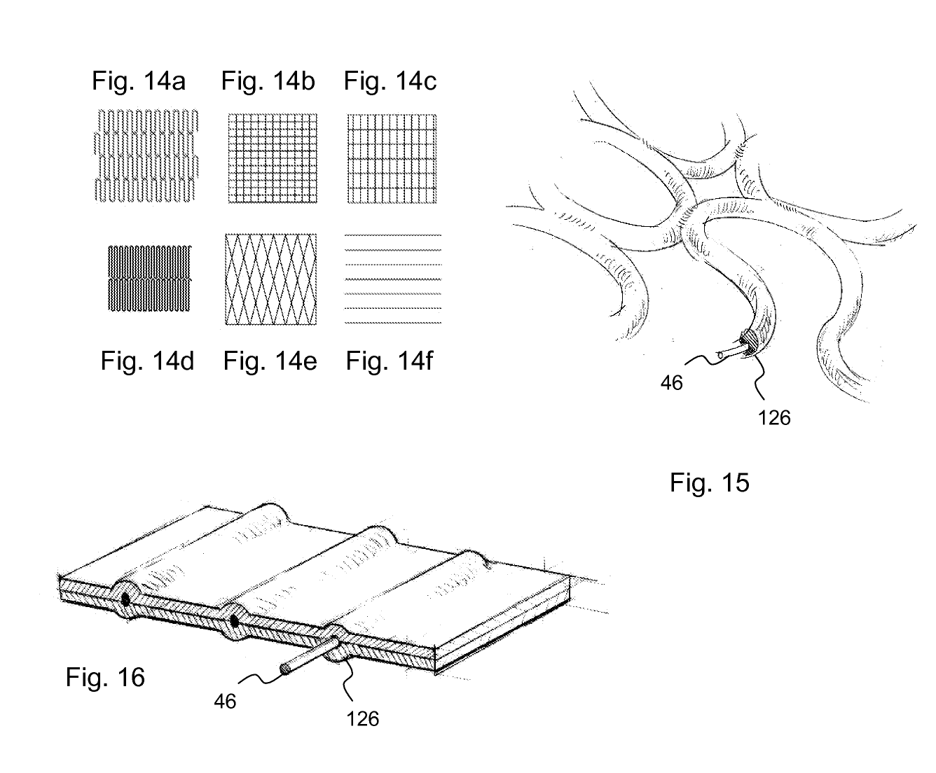

Referring to FIGS. 14a-14f, 15, and 16, various embodiments of the mesh material 46 of FIG. 1 are illustrated. The mesh 46 may include different patterns and/or materials, and may be coated with a polymer or elastomer 126. The coating may be on individual strands of the mesh (FIG. 15) or may be a sheet covering an entire side of the mesh (FIG. 16).

It is understood that various modifications may be made to the preceding embodiments. For example, some portions of the retractor 10 may be formed from a translucent material, such as certain forms of plastic. It is understood that the material may have additional properties, such as being capable of withstanding a high heat sterilization procedure such as autoclaving. The translucent material may enable light to shine through the retractor to illuminate the void 44 and the corresponding surgical area. In some embodiments, the light may scatter within the translucent material to create a relatively large light source.

It is understood that terms such as “side”, “top”, “bottom”, “front”, “back”, “proximal”, and “distal” are relative and may be interchangeable depending on the perspective from which the device of the present disclosure is being viewed. Accordingly, such terms are used for purposes of illustrating and describing various embodiments of the present disclosure and are not intended to be limiting.

The foregoing description of the embodiments of the invention has been presented for the purposes of illustration and description. It is not intended to be exhaustive or to limit the invention to the precise form disclosed. Many modifications and variations are possible in light of the above teaching. For example, various elements of the embodiments described above may be combined with elements of other embodiments, and characteristics of one embodiment may be incorporated into another embodiment. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the invention is intended to encompass within its scope such processes, machines, manufacture, compositions of matter, means, methods, or steps, and it is intended that the scope of the invention not be limited by this detailed description.

What is claimed is:

1. A surgical retractor comprising:
a first wall portion having a first proximal end, a first distal end, first and second edges, a first interior surface, and a first exterior surface, wherein at least a portion of the first wall portion is formed of a flexible material;
a second wall portion having a second proximal end, a second distal end, third and fourth edges, a second interior surface, and a second exterior surface, wherein at least a portion of the second wall portion is formed of a flexible material, and wherein the second wall portion is positioned so that the second interior surface faces the first interior surface;
a first extension sleeve coupled to the first and second wall portions, wherein a longitudinal axis of the first extension sleeve extends from each of the first and second proximal ends to each of the first and second distal ends, respectively; and
a second extension sleeve coupled to the first and second wall portions, wherein a longitudinal axis of the second extension sleeve extends from each of the first and second proximal ends to each of the first and second distal ends, respectively,
wherein the first extension sleeve is movable relative to the second extension sleeve from a first position to a second position to resize a void formed by the first and second wall portions and first and second extension sleeves, the void being open at the first and second proximal ends and the first and second distal ends.

2. The retractor of claim 1 wherein the flexible material is a mesh.

3. The retractor of claim 1 wherein the flexible material is formed from at least one wire.

4. The retractor of claim 1 wherein the flexible material is formed using a polymer material.

5. The retractor of claim 1 wherein the flexible material is at least partially encased in at least one of a polymer or an elastomer shell.

6. The retractor of claim 1 wherein the flexible material is formed using a shape memory alloy.

7. The retractor of claim 1 wherein the first wall portion further includes a rigid portion.

8. The retractor of claim 7 wherein the rigid portion includes a rigid panel.

9. The retractor of claim 7 wherein the rigid portion includes a spline.

10. The retractor of claim 1 wherein the first extension sleeve includes at least first and second grooves for receiving the first and third edges, respectively.

11. A surgical kit comprising:
a retractor having:
first and second wall portions formed at least partially from a flexible material, wherein each of the first and second wall portions includes a proximal end and a distal end; and
first and second extension sleeves coupled to each of the first and second wall portions, wherein a void formed by the first and second wall portions and first and second extension sleeves is open at the first and second proximal ends and the first and second distal ends, and wherein the first extension sleeve is movable relative to the second extension sleeve from a first position to a second position to resize the void; and
a spreader sized for insertion into the void, wherein the spreader includes:
at least one gripping surface for engaging at least one of the first and second wall portions or the first and second extension sleeves; and
at least one handle coupled to the at least one gripping surface, wherein the at least one handle is movable to alter a distance between the first and second wall portions or the first and second extension sleeves.

12. The kit of claim 11 further comprising a light source configured to couple to the retractor.

13. The kit of claim 12 wherein the light source is an optic fiber.

14. A surgical kit comprising:
a deformable mesh body having a proximal end and a distal end, wherein a void formed by the mesh body has openings at the proximal and distal ends; and
a spreader sized for insertion into the mesh body, wherein the spreader includes first and second gripping surfaces for engaging the mesh body and first and second handles coupled to the first and second gripping surfaces, respectively, wherein the first handle is movable relative to the second handle to alter a distance between the first and second gripping surfaces.

15. A surgical retractor comprising:
first and second wall portions formed at least partially from a flexible material, wherein each of the first and second wall portions includes a proximal end and a distal end; and
first and second extension sleeves coupled to each of the first and second wall portions, wherein a void defined by the first and second wall portions and the first and second extension sleeves is open at the first and second proximal ends and the first and second distal ends, and wherein the first extension sleeve is movable relative to the second extension sleeve from a first position to a second position to resize the void.

16. The retractor of claim 15 wherein the first and second extension sleeves each include at least one groove therein for receiving the first and second wall portions.

17. The retractor of claim 15 wherein an inner surface of the first wall portion faces an inner surface of the second wall portion, and wherein the inner surface of the first wall portion is movable relative to the inner surface of the second wall portion due to the flexible material of the first wall portion.

18. The retractor of claim 15 wherein the first extension sleeve is in contact with the second extension sleeve when the first extension sleeve is in the first position.

19. The retractor of claim 15 wherein the first and second wall portions are removable from the first and second extension sleeves.

20. The retractor of claim 15 further comprising a holder coupled to at least one of the first and second extension sleeves and the first and second wall portions.

21. A surgical method comprising:
forming an incision in a tissue at a surgical site;
sequentially dilating the tissue surrounding the surgical site to form a sequentially dilated surgical opening;
inserting a retractor into the sequentially dilated surgical opening; and
separating a first extension sleeve of the retractor from a second extension sleeve of the retractor to enlarge a void defined by the retractor in a first direction, wherein the separating is achieved by applying a force necessary to stretch first and second walls formed at least partially of a flexible material, wherein the first and second walls join the first and second extension sleeves.

22. The method of claim 21 wherein separating the first and second extension sleeves includes inserting a spreader into the void and expanding the spreader.

23. The method of claim 21 further comprising separating the first and second walls of the retractor to enlarge the void in a second direction.
